(19) United States
(12) Patent Application Publication
(10) Pub. No.: US 2025/0018110 A1
(43) Pub. Date: Jan. 16, 2025

(54) INFUSION SET AND ASSOCIATED METHODS

(71) Applicant: Unomedical A/S, Lejre (DK)

(72) Inventors: Omid Mostafaei, Lejre (DK); Lisa Jaunet van Kooten, Copenhagen (DK)

(21) Appl. No.: 18/902,811

(22) Filed: Sep. 30, 2024

Related U.S. Application Data
(63) Continuation of application No. PCT/EP2023/059063, filed on Apr. 5, 2023.
(60) Provisional application No. 63/328,806, filed on Apr. 8, 2022.
(60) Provisional application No. 63/413,360, filed on Oct. 5, 2022.
(60) Provisional application No. 63/413,370, filed on Oct. 5, 2022.

(30) Foreign Application Priority Data
May 23, 2022 (GB) 2207515.4
Nov. 22, 2022 (GB) 2217425.4
Dec. 7, 2022 (GB) 2218352.9

(51) Int. Cl. A61M 5/165; A61M 5/14

(52) U.S. Cl. CPC A61M 5/165; A61M 5/1413; A61M 2005/1657

(57) ABSTRACT

An infusion set for subcutaneous infusion of a therapeutic agent into a patient, the infusion set comprising: an infusion hub comprising: a casing; a cannula for insertion into a patient; a fluid transfer part connected to the casing in fluid communication with the cannula; and, a tubing connector configured to engage the fluid transfer part and connectable to tubing for receiving a therapeutic agent from a pump; an adaptor comprising: an upstream end connectable to the tubing; and, a downstream end connectable to the fluid transfer part; wherein the cannula defines a downstream end of a fluid flow path extending between a pump and the patient via the adaptor; and, wherein the adaptor comprises a filter disposed in the fluid flow path between the upstream end and the downstream end.

### FIELD

The invention relates to an infusion set for subcutaneous infusion of a therapeutic agent into a patient and associated methods. Particularly, though not exclusively, the invention relates to infusion sets for subcutaneous infusion of insulin, heparin, apomorphine, arbidopa, or levodopa and/or levodopa products into a patient.

### BACKGROUND

For patients with diabetes, insulin therapy is often an important part of their treatment, helping to regulate blood sugar levels and store excess glucose for energy. There are two principal modes for delivering insulin. The first mode includes syringes and injector pens, which are used to inject a dose of insulin typically three to four times a day (depending on, inter alia, the type of diabetes and blood sugar levels of the patient). While these devices are simple and low cost, delivering each dose of insulin requires a needle stick. The second mode uses an infusion pump, sometimes called an insulin pump, which delivers controlled doses of insulin throughout the day. An infusion pump can be used to deliver insulin to a patient continuously (basal dose), on demand (bolus dose) or at scheduled intervals. Infusion pumps are more complex and expensive than syringes and pens, though enable improved regulation of blood sugar levels, for example by programmable delivery schedules, and requires fewer needle sticks.

The second mode is known as continuous subcutaneous insulin infusion (CSII) therapy. Infusion pump systems for CSII therapy may be worn by the patient. The systems typically include a combined infusion pump and reservoir fro containing an insulin drug, for example human insulin or analogue insulin, and an insulin infusion set. The infusion set may include a cannula (for example, a polymeric catheter or metal needle) for insertion subcutaneously into the patient and flexible tubing for fluidly connecting the cannula to the reservoir. Once the cannula is inserted into the patient, it may remain in place for a period of time, i.e., days, to allow for continuous delivery of the insulin drug. The current recommended wear time for insulin infusion sets is two to three days, to avoid problems that may arise relating to the infusion set itself or to the infusion site.

However, such problems may still arise within recommended wear times, resulting in early removal of the infusion set and more frequent site rotation across infusion sites (for example buttocks, abdomen and arms).

While problems relating to the infusion set have been well investigated and addressed in recent years, there remains little understanding and few solutions to address problems relating to the infusion site. Problems relating to the infusion site include pain, bleeding, infection, skin irritation, erythema, lipohypertrophy and lipoatrophy. Problems at the infusion site may lead to the build-up of scar tissue, which consequently lowers insulin sensitivity and increases the risk of hypoglycaemia, as well as having a cosmetic impact on patients. All these problems can deter patients from continuing to use their infusion pumps, resulting in poorer patient outcomes.

It is known that problems at the infusion site are a consequence of the immune response to the presence of the cannula and the insulin drug in the body. The immune system responds by activating and progressing the foreign body reaction (FBR)—an inflammatory and fibrotic process that occurs upon introducing a foreign material into the body. In FBR, cells of the immune system identify foreign materials (such as unwanted biological, chemical, or physical species, present in infusible solutions) and attempt to degrade it, or otherwise encapsulate the material by forming a physical barrier to isolate it from the rest of the body. FBR is a problem for increasing wear times of infusion sets in CSII therapy, as the immune system reacts to the inserted cannula and the insulin drug. This limitation prevents realising the full potential of CSII therapy.

Generally, unwanted species, whether biological, chemical or physical, present in infusible solutions have undesirable consequences for patients.

It is an object of embodiments of the invention to provide an improved infusion set that attempts to circumvent FBR, increase wear times of infusion sets, and/or at least mitigate one or more problems associated with known arrangements.

### BRIEF SUMMARY OF THE DISCLOSURE

The invention is defined by the appended claims.

The present disclosure provides an infusion set for subcutaneous infusion of a therapeutic agent into a patient, the infusion set comprising: an infusion hub comprising: a casing; a cannula for insertion into a patient; a fluid transfer part connected to the casing in fluid communication with the cannula; and, a tubing connector configured to engage the fluid transfer part and connectable to tubing for receiving a therapeutic agent from a pump; an adaptor comprising: an upstream end connectable to the tubing; and, a downstream end connectable to the fluid transfer part; wherein the cannula defines a downstream end of a fluid flow path extending between a pump and the patient via the adaptor; and, wherein the adaptor comprises a filter disposed in the fluid flow path between the upstream end and the downstream end.

The present disclosure also provides a method of treating a patient via subcutaneous infusion with a therapeutic agent, the method comprising the steps of: providing an infusion set as described herein; placing the infusion hub at an infusion site of a patient, connecting, via tubing, a pump fluidly connected to a source of a therapeutic agent to the upstream end of the adaptor, connecting the downstream end of the adaptor to the fluid transfer part, priming the tubing with the therapeutic agent, and injecting the patient with the therapeutic agent.

The present disclosure also provides a method of treating a patient via subcutaneous infusion with a therapeutic agent, the method comprising the steps of: providing an infusion set as described herein, connecting, via tubing, a pump fluidly connected to a source of a therapeutic agent to the upstream end of the adaptor, connecting the downstream end of the adaptor to the fluid transfer part, priming the tubing with the therapeutic agent, placing the infusion hub at an infusion site of a patient, and injecting the patient with the therapeutic agent.

In examples, the upstream end of the adaptor comprises a connector for connecting to a connector of the tubing. The connector of the adaptor may be the same as the tubing connector of the infusion hub. This advantageously allows the present infusion sets to be used with existing tubing and pumps.

The present disclosure also provides an adaptor for use in an infusion set for subcutaneous infusion of a therapeutic agent into a patient, the adaptor comprising: an upstream end connectable to tubing, the tubing having a tubing connector connectable to the upstream end and adapted to receive the therapeutic agent from a pump via a pump outlet connector; a downstream end connectable to a fluid transfer part of an infusion hub; and, a filter disposed in a fluid flow path defined between the upstream end and the downstream end. In examples, the upstream end of the adaptor comprises a connector for connecting to a connector of the tubing. The connector of the adaptor may be the same as the tubing connector of the infusion hub. This advantageously allows the present adaptor to be used with existing infusion hubs, tubing and pumps.

In some cases, the infusion set further comprises: tubing connectable between the tubing connector of the infusion hub and a pump; and, a pump for containing and delivering the therapeutic agent to the infusion hub via the tubing.

In some cases, the upstream end of the adaptor comprises a first portion for connecting to the tubing in the fluid flow path. In some cases, the downstream end of the adaptor comprises a second portion for connecting to the fluid transfer part of the infusion hub; and, wherein the filter is disposed in the fluid flow path between the first portion and the second portion inclusive. In some cases, the downstream end of the adaptor is connectable to the fluid transfer part via the tubing connector of the infusion hub.

In some cases, the filter is disposed in the fluid flow path within the first portion, or the second portion.

In some cases, the adaptor comprises tubing connecting the upstream end of the adaptor and the downstream end of the adaptor; and, wherein the filter is disposed in the fluid flow path within the tubing.

In some cases, the tubing has a first section, a second section, and a capsule disposed between the first section and the second section; wherein the filter is disposed within the capsule; and, wherein the fluid flow path extends through the first section, the capsule, and the second section.

In some cases, the adaptor comprises a housing, and wherein the upstream end of the adaptor and the downstream end of the adaptor are integral to the housing. In some cases, the upstream end and the downstream end are part of the same housing. The housing preferably contains the filter.

In some cases, the adaptor comprises a sealing membrane configured to prevent egress of fluid out of the adaptor from the fluid flow path.

In some cases, the sealing membrane is disposed at the upstream end of the adaptor.

In some cases, the filter provides a physical filter medium for removing unwanted species, for example, by size exclusion.

In some cases, the filter provides a chemical filter medium for removing unwanted species, for example, by sorption.

In some cases, the filter is a modular filter comprising first and second sub-filters arranged to allow fluid flow therethrough in series.

In some cases, the first sub-filter comprises a filter material and the second subfilter comprises a further filter material different to the filter material.

In some cases, the filter comprises a foam. The foam may comprise any combination of a cellulose, a polyurethane (Pll), a polyester, a polyether and a collagen. While a foam is described herein, it would be apparent other structures, such as a membrane or a sheet or similar may be used in place of a foam and the properties described in relation to foams apply equally to membranes or sheets.

In some cases, the therapeutic agent comprises any of insulin, heparin, apomorphine, arbidopa, or levodopa and/or levodopa products.

In some cases, the interface of the fluid transfer part comprises a sealing membrane configured to prevent egress of fluid out of the fluid transfer part from the fluid flow path.

In some cases, the cannula may comprise a lumen wall having surface features for maintaining and/or locating the filter within the cannula. The cannula may be a soft polymeric catheter or a metal needle. The cannula may comprise one of a polytetrafluoroethylene (PTFE), a fluorinated ethylene propylene (FEP), a rubber, a polyethylene (PE), a polyurethane (Pll), a polypropylene (PP) or a silicone material. The cannula may be insertable in the patient by an insertion needle.

Infusion sets with filters and associated methods as described herein may be useful in inhibiting FBR at an infusion site, and thereby may avoid problematic occurrences such as coagulation, occlusion and/or inflammation at the infusion site, and/or encapsulation of the cannula. In particular, devices and methods as described herein may be useful in inhibiting FBR at the infusion site in diabetic patients receiving CSII therapy. The infusion site may be a single infusion site in use for an extended period of time, for example at least four days.

Infusion sets with filters and associated methods as described herein may be useful in removing unwanted species from a therapeutic agent before delivery of the therapeutic agent to a patient, for example where unwanted species include preservatives necessarily present in insulin solutions to stabilise and/or sterilise insulin solutions prior to delivery to a patient, but which are cytotoxic.

### BRIEF DESCRIPTION OF THE DRAWINGS

Exemplary infusion sets and associated methods are further described hereinafter with reference to the accompanying drawings, in which:

FIG. 1A is an illustration of an infusion set;

FIG. 1B is a further illustration of the infusion set of FIG. 1A;

FIG. 2A is a side cross-section view of an infusion set, including a first exemplary adaptor;

FIG. 2B is a perspective view of the infusion set of FIG. 2A;

FIG. 2C is a plan cross-section view of the infusion set of FIG. 2A;

FIG. 3A is a side cross-section view of an infusion set, including a second exemplary adaptor;

FIG. 3B is a perspective view of the infusion set of FIG. 3A;

FIG. 3C is a plan cross-section view of the infusion set of FIG. 3A;

FIG. 4A is a side cross-section view of an infusion set, including a third exemplary adaptor;

FIG. 4B is a perspective view of the infusion set of FIG. 4A;

FIG. 4C is a plan cross-section view of the infusion set of FIG. 4A;

FIG. 5A is a side cross-section view of an infusion set, including a fourth exemplary adaptor;

FIG. 5B is a perspective view of the infusion set of FIG. 5A; and,

FIG. 5C is a plan cross-section view of the infusion set of FIG. 5A.

### DETAILED DESCRIPTION

The presently described infusion sets and associated methods have particular application for use with infusion pump systems such as an infusion pump for delivery of a therapeutic agent, such as insulin, heparin or any other liquid therapeutic agents, where the infusion pump includes a fluid pump and a reservoir, and an infusion set having a cannula (typically part of an infusion hub) and tubing for connecting the cannula to the reservoir. The infusion pump may be an insulin pump for CSII therapy, and the therapeutic agent may be an insulin formulation. The presently described infusion sets and associated methods are able to deliver insulin to a patient at a single infusion site over an extended period of time. An extended period of time is to be understood to mean at least four days. More specifically, an extended period of time may include four to seven days, seven or more days, seven to 10 days, and 10 or more days. An extended period of time may include 14 or more days.

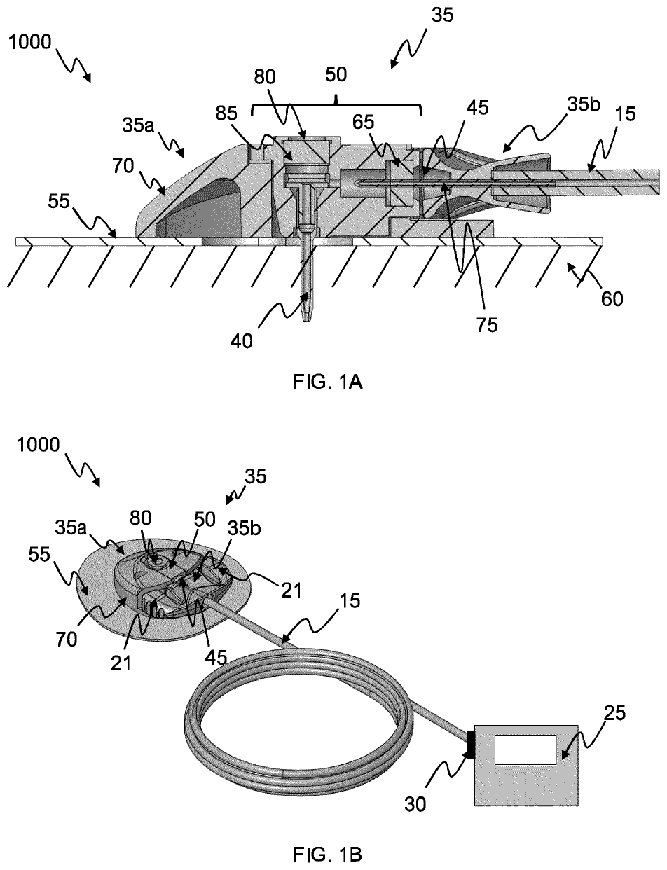

FIGS. 1A and 1 B illustrate an infusion set 1000. The infusion set 1000 includes an infusion hub 35, including a body 35a and a tubing connector 35b. The infusion hub 35 is secured to the skin of a patient by an adhesive patch 55, which maintains a cannula 40 subcutaneously within the sub-dermal fatty tissue of a patient. Tubing 15 connects a pump outlet connector 30 of a pump 25 (the pump 25 containing the therapeutic agent) with the infusion hub 35 via the tubing connector 35b. The body 35a includes a casing 70 and a fluid transfer part 50 which provides a fluid channel. The fluid transfer part 50 is secured within the casing 70 and retains the cannula 40 within the patient. The fluid transfer part 50 may be integral with the casing 70.

The tubing connector 35b engages an interface 45 of the fluid transfer part 50 to connect a downstream end of the tubing 15 to the fluid transfer part 50. The tubing connector 35b includes a releasable connector, in this case a releasable snap-fit joint. As shown in FIG. 1B, the tubing connector 35b has mechanical elements in the form of resiliently deformable arms 21 that engage corresponding mechanical receiving elements on the body 35a of the infusion hub 35, or more specifically, the casing 70 of the body 35a. While the mechanical elements are arranged as a releasable clip in the illustrated tubing connector 35b, it would be apparent that this is not essential. When the tubing connector 35b is secured to the body 35a, a needle 75 of the tubing connector 35b pierces a sealing membrane 65 within the fluid transfer part 50, so that the therapeutic agent can flow through the fluid transfer part 50 and into the patient via the cannula 40.

The function of the fluid transfer part 50 is to allow fluid (e.g. a therapeutic agent) to be transferred through the infusion hub 35 (i.e., from the interface 45 of the fluid transfer part 50 to the cannula 40). As can be seen in FIG. 1A, the fluid transfer part 50 provides a fluid flow path that extends firstly approximately parallel to the plane of the skin surface, and then bends to be approximately perpendicular to the skin surface.

The fluid transfer part 50 has multiple interfaces 45, 80. A sealing membrane 65 seals a first interface 45, and a second sealing membrane 85 is used to seal a second interface 80. While two interfaces 45, 80 are shown, it would be apparent more than two interfaces may be provided as required. Some or all the interfaces may have a sealing membrane secured therein to prevent egress of therapeutic agent from the fluid transfer part 50 through the respective interface.

The cannula 40 is a substantially tubular member for insertion in, and delivering a therapeutic agent to, an infusion site 60. The therapeutic agent includes insulin or an insulin solution. A proximal end of the cannula 40 is fluidly connected to a source of a therapeutic agent, here an infusion pump 25. An opposing, distal end of the cannula 40 is positioned in the infusion site 60, extending to a desired depth to deliver the therapeutic agent. The cannula 40 is any suitable cannula suitable for implantation in a tissue site of a patient, such as a polymeric catheter or metal needle.

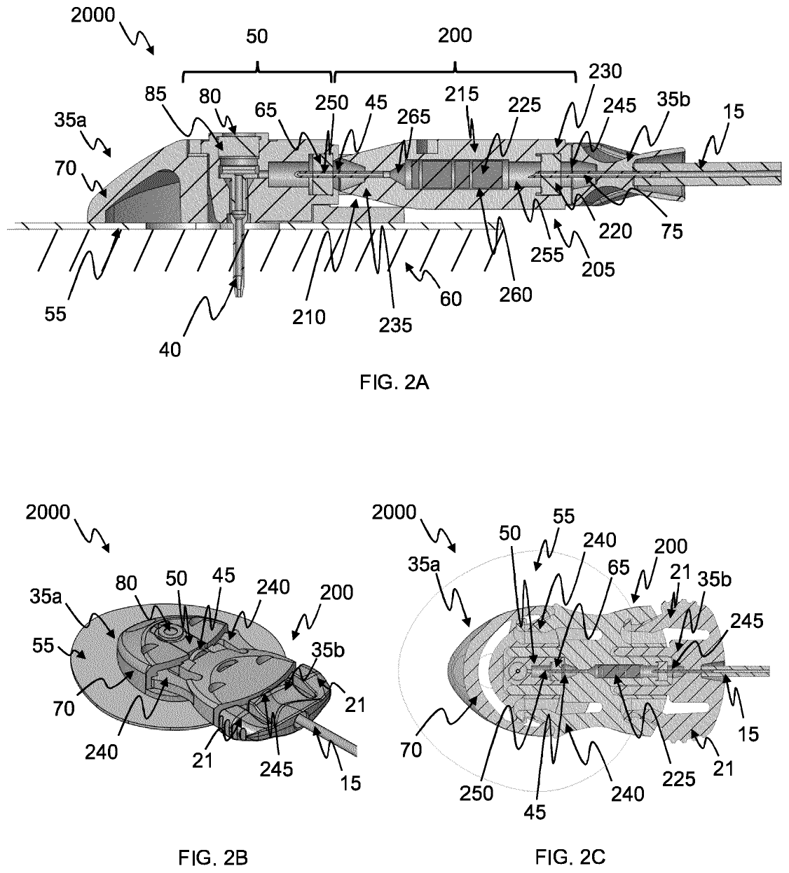

FIGS. 2A to 5C illustrate infusion sets 2000, 3000, 4000, 5000, including an exemplary adaptor 200, 300, 400, 500. The infusion sets of FIGS. 2A to 5C include an infusion hub 35, including a body 35a and a tubing connector 35b, as described above, and further like components are denoted by the same reference numerals. The adaptor 200, 300, 400, 500 is provided between the body 35a and the tubing connector 35b of the infusion hub 35. Each adaptor 200, 300, 400, 500 includes an upstream end 205, 305, 405, 505 connected to tubing 15 via a tubing connector 35b. The tubing 15 is connected to an infusion pump 25 via a pump outlet connector 30 to receive the therapeutic agent as described above. Each adaptor 200, 300, 400, 500 has a downstream end 210, 310, 410, 510 connected to the fluid transfer part 50. Connecting the adaptors 200, 300, 400, 500 in this way means each adaptor 200, 300, 400, 500 forms part of the fluid flow path through which the therapeutic agent is transferred and allows for filtering of the therapeutic agent prior to delivery to the patient.

The tubing connector 35b engages an interface 245, 345, 445, 560 at the upstream end 205, 305, 405, 505 of the adaptors 200, 300, 400, 500 to connect a downstream end of the tubing 15 to the adaptor 200, 300, 400, 500. The tubing connector 35b includes a releasable connector, in this case a releasable snap-fit joint. As shown in FIGS. 2C, 3C, 4C, 5C, the tubing connector 35b has mechanical elements in the form of resiliently deformable arms 21 that engage corresponding mechanical receiving elements at the upstream end 205, 305, 405, 505 of the adaptors 200, 300, 400, 500. The deformable arms 21 of the tubing connector 35b may engage mechanical receiving elements of a first portion 230, 330, 430, 530 of the adaptor 200, 300, 400, 500. While the mechanical elements are arranged as a releasable clip in the illustrated tubing connector 35b, it would be apparent that this is not essential and in some cases the mechanical elements are not releasable. When the tubing connector 35b is secured to the adaptor 200, 300, 400, 500, a needle 75 of the tubing connector 35b pierces a sealing membrane 220, 320, 420, 520 within the upstream end 205, 305, 405, 505 of the adaptors 200, 300, 400, 500 so that the therapeutic agent can flow through the adaptor 200, 300, 400, 500. The sealing membrane 220, 320, 420, 520 prevents egress of the therapeutic agent from the adaptor 200, 300, 400, 500. The sealing membrane 220, 320, 420, 520 may be disposed in a first portion 230, 330, 430, 530 of the adaptor 200, 300, 400, 500.

Further, each adaptor 200, 300, 400, 500 is typically connected to the fluid transfer part 50 of the body 35a via a releasable connector. The adaptor 200, 300, 400, 500 engages an interface 45 of the fluid transfer part 50 to connect a downstream end 210, 310, 410, 510 of the adaptor 200, 300, 400, 500 to the fluid transfer part 50. The adaptor 200, 300, 400, 500 includes a releasable connector, in this case a releasable snap-fit joint. As shown in FIGS. 2C, 3C, 4C, 5C, the adaptor 200, 300, 400, 500 has mechanical elements in the form of resiliently deformable arms 240, 340, 440, 555 that engage corresponding mechanical receiving elements on the body 35a of the infusion hub 35, or more specifically, the casing 70 of the body 35a. While the mechanical elements are arranged as a releasable clip in the illustrated adaptors 200, 300, 400, 500, it would be apparent that this is not essential. When the adaptor 200, 300, 400, 500 is secured to the body 35a, a needle 250, 350, 450, 565 of the adaptor 200, 300, 400, 500 pierces a sealing membrane 65 within the fluid transfer part 50, so that the therapeutic agent can flow through the fluid transfer part 50 and into the patient via the cannula 40. The sealing membrane 65 prevents egress of the therapeutic agent from the fluid transfer part 50.

While the fluid flow path is shown as passing through each adaptor 200, 300, 400, 500 in a substantially linear direction, it would be apparent that this is not essential, and that other configurations of the fluid flow path would be suitable (e.g., including any of an arcuate, a serpentine, or linear sections within any of the components of the adaptor 200, 300, 400, 500). The illustrated adaptors 200, 300, 400, 500 have an elongate structure and the upstream end 205, 305, 405, 505 is disposed at an opposite end to the downstream end 210, 310, 410, 510. The infusion sets 2000, 3000, 4000, 5000 will be explained in more detail below.

FIGS. 2A to 2C illustrate an infusion set 2000, including a first exemplary adaptor 200. The adaptor 200 includes a housing 215 having a first portion 230 at an upstream end 205 of the adaptor 200, and a second portion 235 at a downstream end 210 of the adaptor 200. The first portion 230 and the second portion 235 are integral to the housing 215. The adaptor 200 illustrated in FIG. 2A to 2C has a unitary construction (e.g. with no tubing between the first portion 230 and the second portion 235).

The adaptor 200 forms part of the fluid flow path between the pump 25 and the patient and includes a filter 225 for removing unwanted species from the therapeutic agent prior to delivery of the therapeutic agent to the patient, close to end of the fluid flow path. The filter 225 is located within the fluid flow path of the adaptor 200 in a cavity 260 between the first portion 230 and the second portion 235. The cavity 260 has a geometry substantially corresponding to that of the filter 225. The filter 225 may be cylindrical, conical, a sheet or any functional form in shape. Further, the filter 225 is sized to fit at least the width of the cavity 260, such that any fluid (e.g., a therapeutic agent) in the fluid flow path necessarily passes through the filter 225. The adaptor 200 has an upstream fluid lumen 255 that operatively connects the needle 75 of the tubing connector 35b with the cavity 260. At the other end of the cavity 260, a downstream fluid lumen 265 connects the cavity 260 with a needle 250 of the adaptor 200 that penetrates the sealing membrane 65 of the fluid transfer part 50.

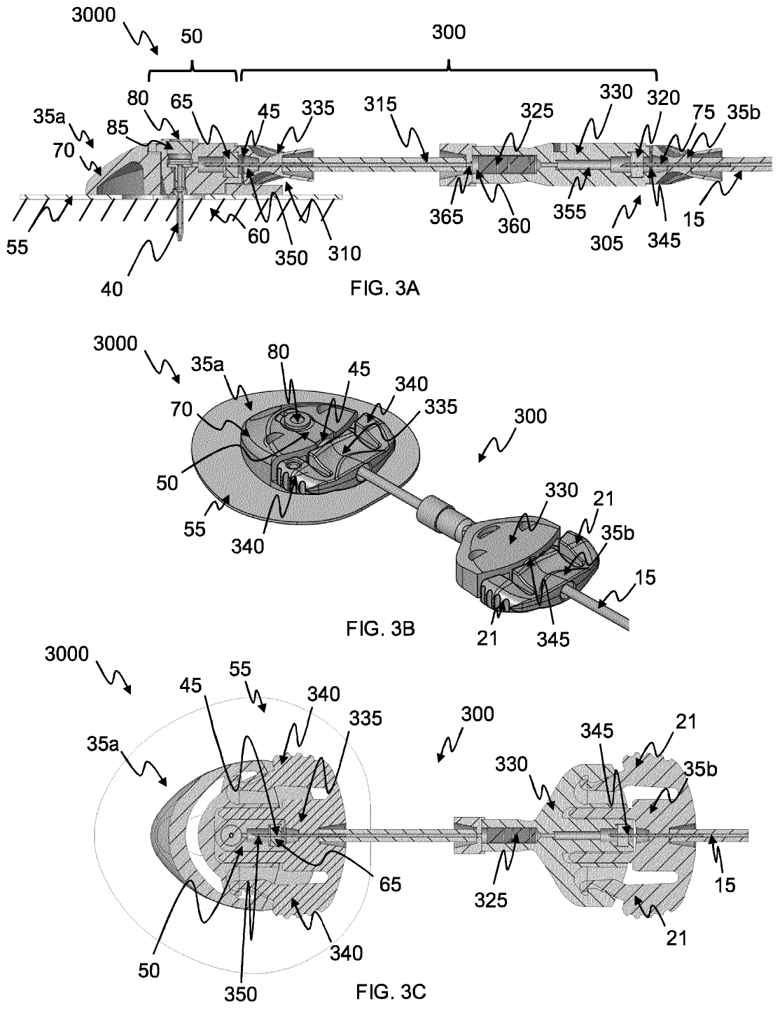

FIGS. 3A to 3C illustrate an infusion set 3000, including a second exemplary adaptor 300. The adaptor 300 includes a first portion 330 located at an upstream end 305 of the adaptor 300. The first portion 330 is connected to a second portion 335 located at a downstream end 310 of the adaptor 300 by a relatively short section of tubing 315. The section of tubing 315 is shorter than the tubing 15 used to connect the pump 25 to the adaptor 300. The tubing 315 has a length of between 1 and 10 cm. The connection between the first portion 330 and tubing 315 may be formed by gluing or welding for example. A person skilled in the art would appreciate that alternate methods of connecting the first portion 330 and the tubing 315 are possible. At the downstream end 310 of the adaptor 300, the tubing 315 is fluidly connected to the second portion 335, preferably by welding or gluing, as explained above.

The adaptor 300 forms part of the fluid flow path between the pump 25 and the patient and includes a filter 325 for removing unwanted species from the therapeutic agent prior to delivery of the therapeutic agent to the patient, close to end of the fluid flow path.

The filter 325 is located within the fluid flow path of the adaptor 300 in a cavity 360 of the first portion 330. The cavity 360 has a geometry substantially corresponding to that of the filter 325. The filter 325 may be cylindrical, conical, a sheet or any functional form in shape. Further, the filter 325 is sized to fit at least the width of the cavity 360, such that any fluid (e.g., a therapeutic agent) in the fluid flow path necessarily passes through the filter 325. The first portion 330 has an upstream fluid lumen 355 that operatively connects the needle 75 of the tubing connector 35b with the cavity 360. At the other end of the cavity 360, a downstream fluid lumen 365 connects the cavity 360 with the tubing 315, and thereby the second portion 335.

The tubing 315 is preferably flexible and may have kink resistant and/or preservative retention properties. It would be apparent that these tubing properties apply to any of the tubing 315, 415, 540, 545, used in the infusion sets 3000, 4000, 5000 described herein.

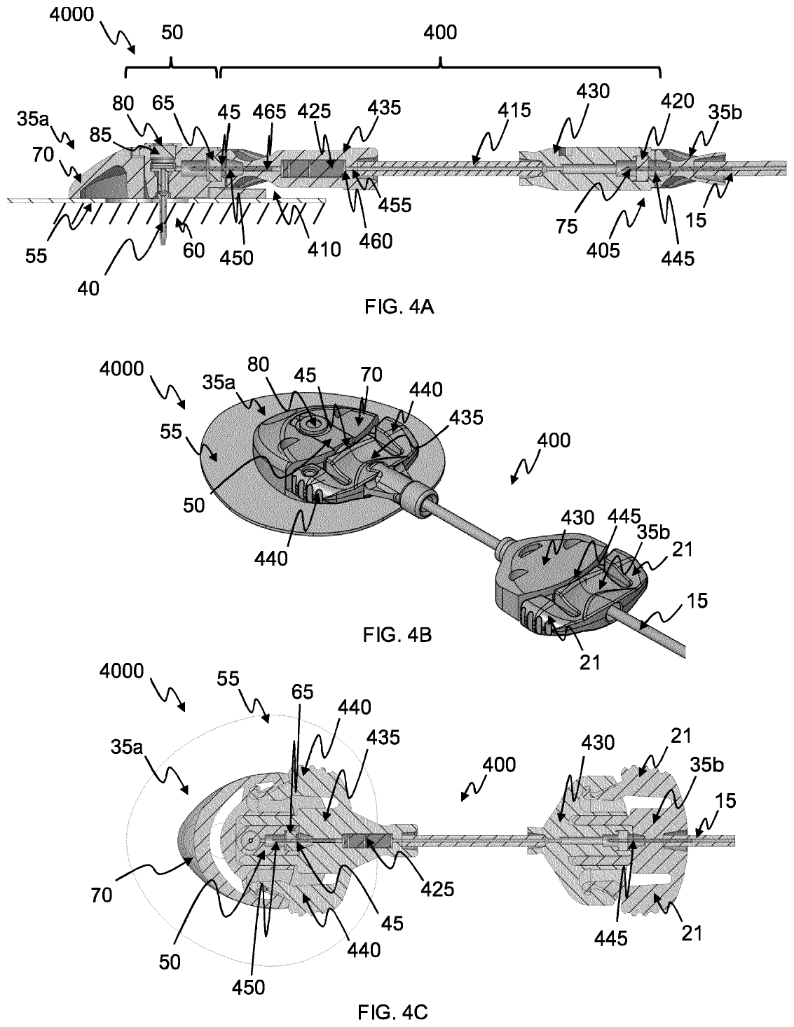

FIGS. 4A to 4C illustrate an infusion set 4000, including a third exemplary adaptor 400. The adaptor 400 includes a first portion 430 located at an upstream end 405 of the adaptor 400 and a second portion 435 located at a downstream end 410 of the adaptor 400. The first portion 430 is connected to the second portion 435 by a section of tubing 415. The tubing 415 is shorter than the tubing 15 used to connect the adaptor 400 to the pump 25. The tubing 415 has a length of between 1-10 cm. The connection between the second portion 435 and tubing 415 may be formed by gluing or welding. A person skilled in the art would appreciate that alternate methods of connecting the second portion 435 and the tubing 415 are possible. At the upstream end 405 of the adaptor 400, the tubing 415 is fluidly connected to the first portion 430, preferably by welding or gluing, as explained above.

The adaptor 400 forms part of the fluid flow path between the pump 25 and the patient and includes a filter 425 for removing unwanted species from the therapeutic agent prior to delivery of the therapeutic agent to the patient, close to end of the fluid flow path. The filter 425 is located within the fluid flow path of the adaptor 400 in a cavity 460 of the second portion 435. The cavity 460 has a geometry substantially corresponding to that of the filter 425. The filter 425 may be cylindrical, conical, a sheet or any functional form in shape. Further, the filter 425 is sized to fit at least the width of the cavity 460, such that any fluid (e.g., a therapeutic agent) in the fluid flow path necessarily passes through the filter 425. The second portion 435 has an upstream fluid lumen 455 that operatively connects the tubing 415 with the cavity 460. At the other end of the cavity 460, a downstream fluid lumen 465 connects the cavity 460 with a needle 450 of the adaptor 400 that penetrates the sealing membrane 65 of the fluid transfer part 50.

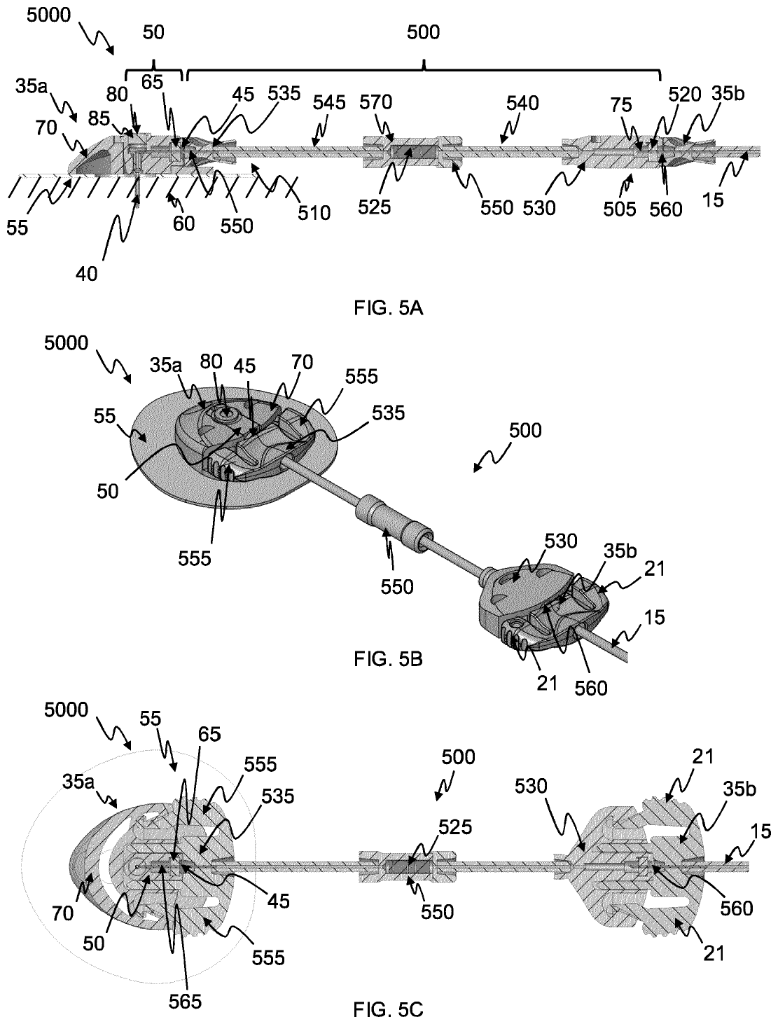

FIGS. 5A to 5C illustrate an infusion set 5000, including a fourth exemplary adaptor 500. The adaptor 500 includes a first portion 530, a second portion 535, a capsule 550 containing a filter 525, a first section of tubing 540 connecting the first portion 530 to the capsule 550, and a second section of tubing 545 connecting the second portion 535 to the capsule 550. While the capsule 550 is provided to secure the filter 525 in position between the first section of tubing 540 and the second section of tubing 545, it would be apparent the filter 525 may be secured between the tubing 540, 545 by other means. The fluid flow path extends through the first tubing section 540, the capsule 550, and second tubing section 545, and the filter 525 is disposed in the fluid flow path within the capsule 550. The filter 525 may be disposed in a cavity 570 of the capsule 550, such that the cavity 570 substantially comprises the geometry of the filter 525. The filter 525 may be cylindrical, conical, a sheet or any functional form in shape. Further, the filter 525 is sized to fit at least the width of the cavity 570, such that any fluid (e.g., a therapeutic agent) in the fluid flow path necessarily passes through the filter 525. The filters 325, 425 described above may be contained in a similar capsule extending from an end of the first portion 330 and the second portion 435 respectively. The capsule may be integrally connected to the respective end of the first portion 330 and the second portion 435.

The first section 540 of tubing is connected to an upstream end of the capsule 550, and the second section 545 of tubing is connected to a downstream end of the capsule 550. While the downstream end 510 is shown at an opposed side of the capsule 550 to the upstream end 505, it would be apparent that this was not essential and other arrangements are possible. The connection between the capsule 550 and the first 540 and second 545 sections of tubing may be formed by gluing or welding as explained above. A person skilled in the art would appreciate that alternate methods of connecting the capsule 550 and the first 540 and second 545 sections of tubing are possible.

The infusion sets 2000, 3000, 4000, 5000 described above each include a filter 225, 325, 425, 525. The filter is intended to remove, for example by filtration, unwanted species present in the therapeutic agent to circumvent FBR. Used herein, unwanted species is to be understood to mean one or more species which may be present in the therapeutic agent, for example by design or accident, and which may be undesirable to remain in the therapeutic agent at the point of delivery to the infusion site. In particular, the filter may remove unwanted species that occur in insulin solutions. Such unwanted species may be particulate and/or molecular in nature. Examples of particulate unwanted species include plastic particles, dust and insulin agglomerates, which have been produced during manufacture, storage, sterilization, or handling of the infusion set and/or the insulin solution. Examples of molecular unwanted species include preservatives commonly used in insulin solutions, such as phenol, cresol (particularly m-cresol), benzyl alcohol, benzalkonium chloride, cetrimide, chlorobutanol, chlorhexidine, chlorocresol, hydroxy benzoates, phenethyl alcohol, phenoxyethanol and phenylmercuric nitrate.

The filter may include any filter material capable of removing one or more unwanted species from the therapeutic agent. To remove particulate unwanted species, the filter material may provide a physical filter medium for removing unwanted species by size exclusion, including whereby the filter material functions as a molecular sieve. Additionally, or alternatively, to remove molecular unwanted species, the filter material may provide a chemical filter medium for removing unwanted species by sorption, for example by adsorption or ion exchange, whereby the filter material binds with the molecular unwanted species to retain them within the filter. The filter material has a plurality of passageways, for example pores (i.e., interconnected hollow voids), extending therethrough to allow fluid flow through the filter.

The filter may be a modular filter including first and second sub-filters arranged to allow fluid flow therethrough in series for progressively removing different unwanted species from the therapeutic agent, for example unwanted species of varying sizes and/or varying molecular composition. Accordingly, the first sub-filter may include a filter material different to that of the second sub-filter. The filter may similarly include a third, a fourth and so on sub-filters.

Suitable filter materials include foams made of a cellulose, a polyurethane, a polyester, a polyether, a collagen or the like. The foam includes a plurality of passageways in the form of interconnected pores extending therethrough to allow fluid flow through the filter. The foam may be any foam capable of removing particulate unwanted species from an insulin solution. The foam may remove particulate unwanted species from an insulin solution by a size exclusion process. While a foam is described herein, it would be apparent other structures, such as a membrane or a sheet or similar may be used in place of a foam and the properties described in relation to foams apply equally to membranes or sheets.

Particularly suitable filter materials are disclosed in earlier patent applications, including U.S. Pat. Nos. 4,083,906 and 11,197,949, the contents of which are incorporated herein by reference.

Suitable filter materials include an ion-exchange resin, including functionalised porous or gel polymers, which may remove unwanted species from an insulin solution by a gel permeation chromatography process. Moreover, gel polymers may be used to coat passageways in the filter material.

Generally, the filter material may be selected to have at least one material property that may facilitate the infusion of insulin at a single infusion site over an extended period of time, and thereby increase wear times, for example at least four days, including four to seven days, seven or more days, seven to 10 days, 10 or more days, and 14 or more days.

Advantageously, each adaptor 200, 300, 400, 500 can be retrofitted to the infusion set 1000 (as shown in FIGS. 1A and 1 B). The adaptor 200, 300, 400, 500 can be retrofitted by attaching the adaptor 200, 300, 400, 500 to the tubing connector 35b at an upstream end 205, 305, 405, 505, and fluid transfer part 50 of the body 35a at a downstream end 210, 310, 410, 510, providing an improved infusion set 2000, 3000, 4000, 5000. As such, a patient can receive the benefits of the presently described infusions sets 2000, 3000, 4000, 5000 as explained below, without having to replace their existing infusion set.

Advantageously, the filter 225, 325, 425, 525 of the adaptors 200, 300, 400, 500 removes, for example by filtration, unwanted species present in the therapeutic agent, thereby circumventing a foreign body reaction (FBR).

Advantageously, as the first portion 230, 330, 430, 530 is configured to engage the existing tubing connector 35b and the second portion 235, 335, 435, 535 is configured to engage the existing fluid transfer part 50 of the body 35a, the caregiver or patient simply needs to connect the adaptor 200, 300, 400, 500 to the body 35a (which will typically be mounted at the infusion site 60), before connecting their existing tubing connector 35b and pump 25 to the adaptor 200, 300, 400, 500.

Advantageously, where the first portion 230 and the second portion 235 of the adaptor 200 are integral to the housing 215, the physical size of the adaptor 200 is minimised and the number of additional components added to the infusion set 1000 is reduced, whilst providing the benefits explained herein.

Advantageously, where the adaptor 300, 400, 500 comprises tubing 315, 415, 545, 540, the tubing 315, 415, 545, 540 of the adaptor 300, 400, 500 provides some flexibility in the precise orientation of the first portion 330, 430, 530 when connected to the body 35a of the infusion hub 35.

Advantageously, where the adaptor 300, 400, 500 comprises tubing 315, 415, 545, 540, the flexible configuration of the tubing 315, 415, 545, 540 increases the durability of the adaptor 300, 400, 500 and thereby the infusion set 3000, 4000, 5000 when in use.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example described herein unless incompatible therewith. All the features disclosed in this specification (including any accompanying claims, abstract and drawings), and/or all the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Certain embodiments are described in the following clauses:

1. An infusion set for subcutaneous infusion of a therapeutic agent into a patient, the infusion set comprising:
an infusion hub comprising:
a casing;
a cannula for insertion into a patient;
a fluid transfer part, connected to the casing and in fluid communication with the cannula; and,
a tubing connector configured to engage the fluid transfer part and connectable to tubing for receiving a therapeutic agent from a pump;
an adaptor comprising:
an upstream end connectable to the tubing; and,
a downstream end connectable to the fluid transfer part;
wherein the cannula defines a downstream end of a fluid flow path extending between a pump and the patient via the adaptor; and,
wherein the adaptor comprises a filter disposed in the fluid flow path between the upstream end and the downstream end.
   2. An infusion set according to clause 1, wherein the upstream end of the adaptor comprises a first portion for connecting to the tubing in the fluid flow path; and, the downstream end of the adaptor comprises a second portion for connecting to the fluid transfer part; and,
wherein the filter is disposed in the fluid flow path between the first portion and the second portion inclusive.
   3. An infusion set according to clause 2, wherein the filter is disposed in the fluid flow path within the first portion or the second portion.
   4. An infusion set according to clause 2, wherein the adaptor comprises tubing connecting the upstream end of the adaptor and the downstream end of the adaptor; and, wherein the filter is disposed in the fluid flow path within the tubing.
   5. An infusion set according to clause 4, wherein the tubing has a first section, a second section, and a capsule disposed between the first section and the second section;
wherein the filter is disposed within the capsule; and,
wherein the fluid flow path extends through the first section, the capsule, and the second section.
   6. An infusion set according to clause 2, wherein the adaptor comprises a housing, and wherein the upstream end of the adaptor and the downstream end of the adaptor are integral to the housing.
   7. An infusion set according to any one of the preceding clauses, wherein the adaptor comprises a sealing membrane configured to prevent egress of fluid out of the adaptor from the fluid flow path.
   8. An infusion set according to clause 7, wherein the sealing membrane is disposed at the upstream end of the adaptor.

1. An infusion set for subcutaneous infusion of a therapeutic agent into a patient, the infusion set comprising:
an infusion hub comprising:
a casing;
a cannula for insertion into a patient;
a fluid transfer part connected to the casing in fluid communication with the cannula; and,
a tubing connector configured to engage the fluid transfer part and connectable to tubing for receiving a therapeutic agent from a pump;
an adaptor comprising:
an upstream end connectable to the tubing; and,
a downstream end connectable to the fluid transfer part;
wherein the cannula defines a downstream end of a fluid flow path extending between a pump and the patient via the adaptor; and,
wherein the adaptor comprises a filter disposed in the fluid flow path between the upstream end and the downstream end.

2. An infusion set according to claim 1, wherein the upstream end of the adaptor comprises a first portion for connecting to the tubing in the fluid flow path; and, the downstream end of the adaptor comprises a second portion for connecting to the fluid transfer part; and,
wherein the filter is disposed in the fluid flow path between the first portion and the second portion inclusive.

3. An infusion set according to claim 2, wherein the filter is disposed in the fluid flow path within the first portion or the second portion.

4. An infusion set according to claim 2, wherein the adaptor comprises tubing connecting the upstream end of the adaptor and the downstream end of the adaptor; and, wherein the filter is disposed in the fluid flow path within the tubing.

5. An infusion set according to claim 4, wherein the tubing has a first section, a second section, and a capsule disposed between the first section and the second section;
wherein the filter is disposed within the capsule; and,
wherein the fluid flow path extends through the first section, the capsule, and the second section.

6. An infusion set according to claim 2, wherein the adaptor comprises a housing, and wherein the upstream end of the adaptor and the downstream end of the adaptor are integral to the housing.

7. An infusion set according to any preceding claim, wherein the adaptor comprises a sealing membrane configured to prevent egress of fluid out of the adaptor from the fluid flow path.

8. An infusion set according to claim 7, wherein the sealing membrane is disposed at the upstream end of the adaptor.

9. An infusion set according to any one of the preceding claims, wherein the filter provides a physical filter medium for removing unwanted species by size exclusion.

10. An infusion set according to any one of the preceding claims, wherein the filter provides a chemical filter medium for removing unwanted species by sorption.

11. An infusion set according to any one of the preceding claims, wherein the filter is a modular filter comprising first and second sub-filters arranged to allow fluid flow therethrough in series.

12. An infusion set according to claim 11, wherein the first sub-filter comprises a filter material and the second sub-filter comprises a further filter material different to the filter material.

13. An infusion set according to any one of the preceding claims, wherein the filter comprises a foam or a membrane or a sheet comprising a cellulose, a polyurethane (Pll), a polyester, a polyether and/or a collagen.

14. An infusion set according to any one of the preceding claims, further comprising: tubing connectable between the fluid transfer part of the infusion hub and a pump; and, a pump for containing and delivering the therapeutic agent to the infusion hub via the tubing.

15. A method of treating a patient via subcutaneous infusion with a therapeutic agent, the method comprising the steps of:
providing an infusion set according to any preceding claim;
placing the infusion hub at an infusion site of a patient,
connecting, via tubing, a pump fluidly connected to a source of a therapeutic agent to the upstream end of the adaptor,
connecting the downstream end of the adaptor to the fluid transfer part, priming the tubing with the therapeutic agent, and
injecting the patient with the therapeutic agent.

16. A method of treating a patient via subcutaneous infusion with a therapeutic agent, the method comprising the steps of:
providing an infusion set according to any of claims 1 to 14,
connecting, via tubing, a pump fluidly connected to a source of a therapeutic agent to the upstream end of the adaptor,
connecting the downstream end of the adaptor to the fluid transfer part, priming the tubing with the therapeutic agent,
placing the infusion hub at an infusion site of a patient, and
injecting the patient with the therapeutic agent.

17. An adaptor for use in an infusion set as described in any of claims 1 to 14, the adaptor comprising:
an upstream end connectable to tubing, the tubing having a tubing connector connectable to the upstream end and adapted to receive the therapeutic agent from a pump via a pump outlet connector;
a downstream end connectable to the fluid transfer part of an infusion hub; and, a filter disposed in a fluid flow path defined between the upstream end and the downstream end.

18. An adaptor according to claim 17, wherein the upstream end of the adaptor comprises a first portion for connecting to the tubing connector of the tubing in the fluid flow path; and, the downstream end of the adaptor comprises a second portion for connecting to the fluid transfer part of the infusion hub; and,
wherein the filter is disposed in the fluid flow path between the first portion and the second portion inclusive.

19. An adaptor according to claim 18, wherein the filter is disposed in the fluid flow path within the first portion or the second portion.

20. An adaptor according to claim 18, comprising a tubing connecting the upstream end of the adaptor and the downstream end of the adaptor; and,
wherein the filter is disposed in the fluid flow path within the tubing.

21. An adaptor according to claim 20, wherein the tubing has a first section, a second section, and a capsule disposed between the first section and the second section;
wherein the filter is disposed within the capsule; and,
wherein the fluid flow path extends through the first section, the capsule, and the second section.

22. An adaptor according to claim 18, comprising a housing, and wherein the upstream end of the adaptor and the downstream end of the adaptor are integral to the housing.

23. An adaptor according to any one of claims 17 to 22, wherein the adaptor comprises a sealing membrane configured to prevent egress of fluid out of the adaptor from the fluid flow path.

24. An adaptor according to claim 23, wherein the sealing membrane is disposed at the upstream end of the adaptor.

25. An adaptor according to any one of claims 17 to 24, wherein the filter provides a physical filter medium for removing unwanted species by size exclusion.

26. An adaptor according to any one of claims 17 to 25, wherein the filter provides a chemical filter medium for removing unwanted species by sorption.

27. An adaptor according to any one of claims 17 to 26, wherein the filter is a modular filter comprising first and second sub-filters arranged to allow fluid flow therethrough in series.

28. An adaptor according to claim 27, wherein the first sub-filter comprises a filter material and the second sub-filter comprises a further filter material different to the filter material.

29. An adaptor according to any one of claims 17 to 28, wherein the filter comprises a foam or a membrane or a sheet comprising a cellulose, a polyurethane (Pll), a polyester, a polyether and/or a collagen.
